(19) United States
(12) Patent Application Publication
(10) Pub. No.: US 2020/0245970 A1
(43) Pub. Date: Aug. 6, 2020

(54) PRESCRIPTIVE GUIDANCE FOR ULTRASOUND DIAGNOSTICS

(71) Applicant: Bay Labs, Inc., San Francisco, CA (US)

(72) Inventors: Charles Cadieu, Menlo Park, CA (US); Ali Chaudhry, San Francisco, CA (US); Sara Guttas, San Francisco, CA (US); Ha Hong, San Ramon, CA (US); Ching Hsieh, San Francisco, CA (US); Kilian Koepsell, San Francisco, CA (US); Nripesh Parajuli, San Francisco, CA (US); Nicolas Poilvert, Seattle, WA (US)

(21) Appl. No.: 16/264,310

(22) Filed: Jan. 31, 2019

(51) Int. Cl. A61B 8/00; G06T 7/00

(52) U.S. Cl. CPC A61B 8/42; G06T 7/0002; G06T 2207/30168; G06T 2207/10132; A61B 8/463

(57) ABSTRACT

A method for prescriptively guiding an operator of an ultrasound imaging probe includes acquiring a sequence of image clips of a target organ utilizing an ultrasound imaging probe, determining a particular view of the target organ expressed by the acquired image clips and assessing a quality of each of the acquired image clips in respect to the particular view. On condition that the assessed quality falls below a pre-determined satisfactory quality for the particular view, a prescriptive movement of the probe is computed that has been pre-determined to produce quality improvement of a subsequently acquired image clip of the target organ for the particular view. Then, responsive to determining that the prescriptive movement has been computed a threshold number of times in connection with the sequence, the prescriptive movement is displayed concurrently with a display of subsequently acquired image clips of the target organ.

### BACKGROUND OF THE INVENTION

### Field of the Invention

The present invention relates to ultrasound imaging and more particularly to ultrasound image acquisition.

### Description of the Related Art

Ultrasound imaging, also known as sonography, is a medical imaging technique that employs high-frequency sound waves to view three-dimensional structures inside the body of a living being. Because ultrasound images are captured in real-time, ultrasound images also show movement of the internal organs of the body as well as blood flowing through the blood vessels of the human body and the stiffness of tissue. Unlike x-ray imaging, ultrasound imaging does not involve ionizing radiation thereby allowing prolonged usage of ultrasound imaging without threatening tissue and internal organ damage from prolonged radiation exposure.

To acquire ultrasound imagery, during an ultrasound exam, a transducer, commonly referred to as a probe, is placed directly on the skin or inside a body opening. A thin layer of gel is applied to the skin so that the ultrasound waves are transmitted from the transducer through the medium of the gel into the body. The ultrasound image is produced based upon a measurement of the reflection of the ultrasound waves off the body structures. The strength of the ultrasound signal, measured as the amplitude of the detected sound wave reflection, and the time taken for the sound wave to travel through the body provide the information necessary to compute an image.

Compared to other prominent methods of medical imaging, ultrasound presents several advantages to the diagnostician and patient. First and foremost, ultrasound imaging provides images in real-time. As well, ultrasound imaging requires equipment that is portable and can be brought to the bedside of the patient. Further, as a practical matter, the ultrasound imaging equipment is substantially lower in cost than other medical imaging equipment, and as noted, does not use harmful ionizing radiation. Even still, the production of quality ultrasound images remains highly dependent upon a skilled operator.

In this regard, depending upon the portion of the body selected for imaging, the skilled operator must know where to initially place the ultrasound probe. Then, the skilled operator must know how to spatially orient the probe and finally, the skilled operator must know where to move the probe so as to acquire the desired imagery. Generally, the ultrasound operator is guided in the initial placement, orientation and movement of the probe based upon the visual feedback provided by the imagery produced during the ultrasound. Thus, essentially, the navigation of the probe is a manual process consisting of iterative trial and error. Plainly, then, the modern process of ultrasound navigation is not optimal.

### BRIEF SUMMARY OF THE INVENTION

Embodiments of the present invention address deficiencies of the art in respect to ultrasound diagnostics and provide a novel and non-obvious method, system and computer program product for prescriptively guiding an operator of an ultrasound imaging probe. In an embodiment of the invention, a method for prescriptively guiding an operator of an ultrasound imaging probe includes acquiring a sequence of image clips of a target organ of a subject utilizing an ultrasound imaging probe of an ultrasound diagnostic system, determining a particular view of the target organ expressed by the acquired image clips and assessing a quality of each of the acquired image clips in respect to the particular view.

On the condition that the assessed quality of the acquired image clip falls below a pre-determined satisfactory quality for the particular view, a prescriptive movement of the ultrasound imaging probe is computed in memory of the ultrasound diagnostic system that has been pre-determined to produce quality improvement of a subsequently acquired image clip of the target organ for the particular view. Then, in response to a determination that the prescriptive movement has been computed a threshold number of times in connection with the sequence of image clips, the prescriptive movement is displayed in a user interface of the ultrasound diagnostic system concurrently with a display in the user interface of subsequently acquired image clips of the target organ.

In one aspect of the embodiment, the method further includes inserting each computed prescriptive movement for a corresponding one of the acquired image clips into a first in first out queue of fixed size and displaying the prescriptive movement in the user interface in response to a determination that the prescriptive movement is present in a threshold number of entries of the first in first out queue. Optionally, the inserting of a computed prescriptive movement occurs only when a probability associated with the computed prescriptive movement exceeds a threshold value. In another aspect of the embodiment, the selected one of the prescriptive movement is shown as a directive to rotate the ultrasound imaging probe without changing position of the ultrasound imaging probe, or as a changing of position of the ultrasound imaging probe in a specified direction or as a changing of a pitch of the ultrasound imaging probe without changing position of the ultrasound imaging probe.

In another embodiment of the invention, a data processing system is configured for prescriptively guiding an operator of an ultrasound imaging probe. The system includes a computer with memory and at least one processor, a display coupled to the computer, beamformer circuitry coupled to the computer and the display and an ultrasound imaging probe comprising a transducer connected to the beamformer circuitry. Of note, a prescriptive guidance module executes in the memory of the computer. The module includes program code enabled upon execution by the processor of the computer to acquire a sequence of image clips of a target organ of a subject utilizing the ultrasound imaging probe, to determine a particular view of the target organ expressed by the acquired image clips, to assess a quality of the acquired image clips in respect to the particular view, and on condition that the assessed quality of the acquired image clips falls below a pre-determined satisfactory quality for the particular view, to compute in memory of the ultrasound diagnostic system for each of the acquired image clips a prescriptive movement of the ultrasound imaging probe pre-determined in order to produce quality improvement of a subsequently acquired image clip of the target organ for the particular view, and, in response to a determination that the prescriptive movement has been computed a threshold number of times in connection with the sequence of image clips, to display the prescriptive movement in a user interface of the ultrasound diagnostic system concurrently with a display in the user interface of subsequently acquired image clips of the target organ.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is a pictorial illustration of a process for prescriptively guiding an operator of an ultrasound imaging probe;

FIG. 2 is a schematic illustration of a data processing system configured for prescriptively guiding an operator of an ultrasound imaging probe; and,

FIG. 3 is a flow chart illustrating a process for prescriptively guiding an operator of an ultrasound imaging probe.

### DETAILED DESCRIPTION OF THE INVENTION

Embodiments of the invention provide for prescriptively guiding an operator of an ultrasound imaging probe. In accordance with an embodiment of the invention, a sequence of image clips of a target organ of a subject are acquired utilizing an ultrasound imaging probe of an ultrasound diagnostic system. A particular view is determined based upon the image clips and the target organ and a determination is made as to the sufficient quality of each of the image clips based upon the particular view. To the extent that the determined quality of each of the image clips is below the sufficient quality, a prescriptive movement of the ultrasound imaging probe is computed in memory of the ultrasound diagnostic system for each of the image clips. In response to a determination that the prescriptive movement has been computed a threshold number of times in connection with the sequence of image clips, the prescriptive movement is then displayed in a display of the ultrasound diagnostic system.

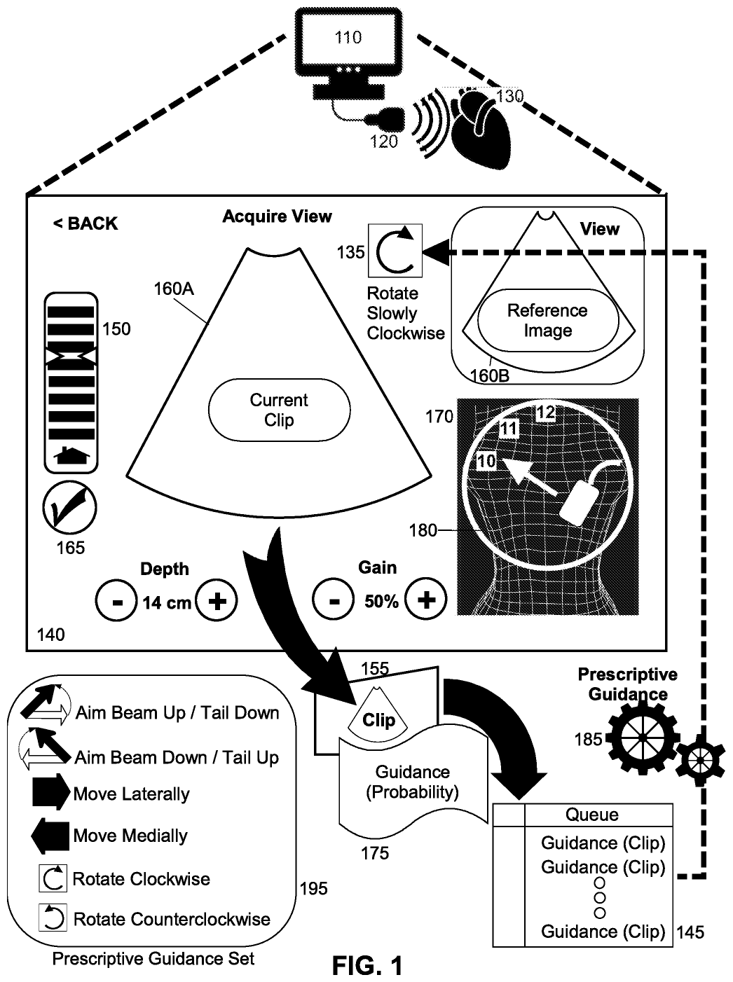

In further illustration, FIG. 1 pictorially shows a process for prescriptively guiding an operator of an ultrasound imaging probe. As shown in FIG. 1, an ultrasound imaging system 110 with ultrasound imaging probe 120 conducts an ultrasound imaging operation in order to image a target organ 130. A user interface 140 to the ultrasound imaging system 110 presents a contemporaneous display 160A of a video clip 155 acquired by the ultrasound imaging probe 120 of the target organ 130. A quality meter 150 is disposed in the user interface 140 and indicates a sliding scale of quality of the video clip in the contemporaneous display 160A relative to a known view sought to be acquired for the target organ 130. For example, in connection with the imaging of a heart, the known view may include a parasternal long axis view, a parasternal short axis view, an apical two, three, four or five chamber view or a subcoastal view, to name a few prospective views. To the extent that the video clip 155 is determined to have a corresponding quality value 175 that meets or exceeds a threshold quality for the specified view, a success icon 165 is displayed in connection with the quality meter 150. As well, a previously acquired, ideal, reference image 160B of the target organ 130 according to the known view is displayed in the user interface 140.

The video clip 155 is than processed in order to determine a quality which is then correlated to a probability of recommending particular prescriptive guidance 175 in order to produce an improved quality of imaging of the target organ 130 for the known view. In this regard, the prescriptive guidance 175 may be selected from amongst different pre-determined movements 195 of the ultrasound imaging probe 120 in order to remediate sub-standard quality for an acquired video clip. Those movements 195 include, by way of example, the rotation of the ultrasound imaging probe 120 either in a clockwise or counter-clockwise direction, the movement of the ultrasound imaging probe 120 laterally away from the sternum, or medially towards the sternum, and the aiming of the imaging beam downwards or upwards by tilting the tail of the ultrasound imaging probe 120 upwards or downwards, respectively, slide downward, rock towards the indicator, rock away from the indicator, tilting the tail medially, or tilting the tail laterally, to name only a few examples.

The prescriptive guidance 175 is then inserted into a first in first out queue 145 in association with the video clip 155 such that a least recent entry inserted into the queue 145 is removed upon inserting a most recent entry. Prescriptive guidance logic 185 then monitors the entries in the queue 145. When, the prescriptive guidance logic 185 determines that a threshold number of entries in the queue 145 exist for the prescriptive guidance 175, an icon 135 representative of the prescriptive guidance 175 is displayed in the user interface 140 and remains displayed in the user interface 140 for so long as the threshold number of entries in the queue 145 remain for the prescriptive guidance 175. When a threshold number of entries in the queue 145 for the prescriptive guidance 175 no longer exist, the icon 135 is removed from the user interface 140 in favor of either an icon for new prescriptive guidance with a corresponding threshold number of entries in the queue 145, or no icon when only a threshold number of null entries corresponding to no prescriptive guidance exist in the queue 145.

Optionally, an additional portion 170 of the user interface 140 may be provided displaying an iconic image of a portion of a body in which the target organ 130 resides and a recommended movement of the ultrasound imaging probe 120 relative to the iconic image in order to achieve the specified view for the target organ 130. The additional portion of the user interface 170 includes a spatial orientation indicator 180 superimposed upon the iconic image of the portion of the body in which the target organ 130 resides. arranged as a clock angle indicator with twelve angularly equidistant positions. In this regard, only a relevant number of the clock angle positions are shown, as can be seen in FIG. 1, which positions are selected as a range towards which the ultrasound imaging probe 120 is known to move in order to acquire imagery of the specified view for the target organ 130. In this way, the combination of the recommended movement and the spatial orientation indicator 180 provide quick visual guidance to the operator of the ultrasound imaging system 110 in order to achieve a video clip of sufficient quality for the selected view.

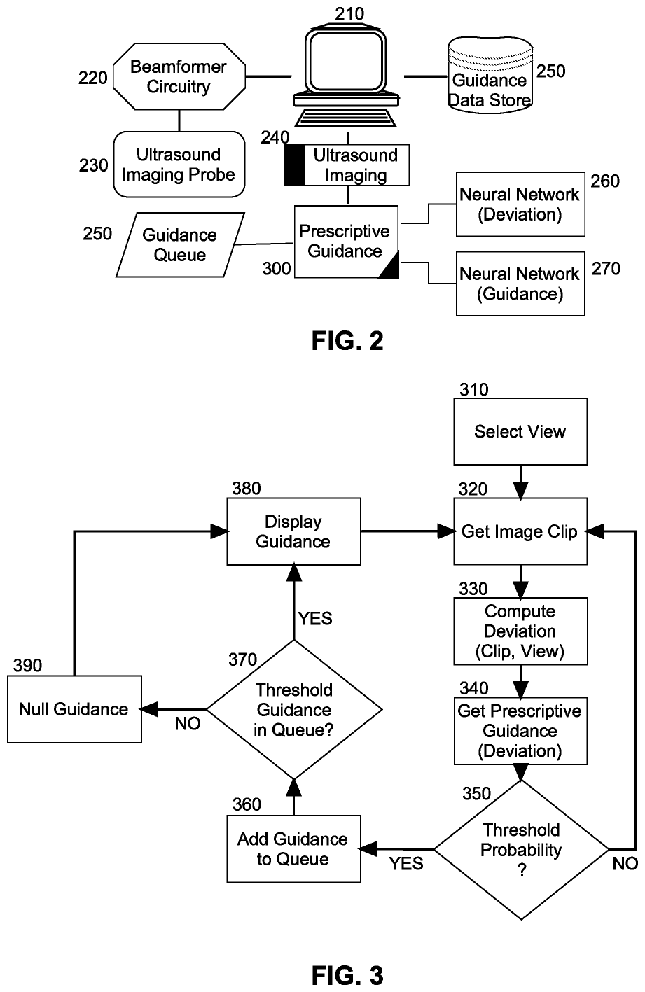

The process described in connection with FIG. 1 may be implemented in a ultrasound diagnostics data processing system. In further illustration, FIG. 2 schematically shows a data processing system configured for prescriptively guiding an operator of an ultrasound imaging probe. The system includes a host computing system 210 that includes a computer with at least one processor, memory and a display. The host computing system 210 also includes a data store 250. The host computing system 210 yet further is coupled to an ultrasound imaging system 220 adapted to store in memory, ultrasound imagery acquired through the placement of an imaging wand 230 proximate to a target organ of interest in a mammalian subject by operation of beamformer circuitry 220.

Importantly, the host computing system 210 is communicatively coupled to fixed storage (not shown), either locally or remotely (“in the cloud”) storing therein a neural network and a programmatic interface to the neural network. The neural network is trained to characterize one or more features of the target organ, for example an ejection fraction value of a heart, or the presence or absence of aortic stenosis. To do so, video clip imagery of a specified view of the target organ acquired by the ultrasound imaging system 220 is provided to the neural network which in turn accesses the programmatic interface so that the neural network may then output the characterization for the video clip imagery along with an indication of confidence in that characterization. The ultrasound imaging system 220 in turn renders on the display of the host computing system 210 not only the video clip imagery, but also the characterization and optionally, the indication of confidence.

In accordance with an embodiment of the invention, a prescriptive guidance module 300 is included with the ultrasound imaging application 220. The module 300 includes computer program instructions enabled, upon execution in the host computing system 210, to receive an acquired video clip image of a target organ according to a specified view of the target organ, and a corresponding determination of quality of the acquired video clip image. The program code is further enabled to correlate the acquired video clip image with a deviation from an ideal image through the use of neural network 260. In this regard, the deviation computed through the use of neural network 260 can be expressed in terms of a location of the ultrasound imaging probe 230 relative to an ideal location—e.g. left of ideal, right of ideal, above ideal or below ideal. As well, the location can be indeterminate or ideal.

The program code yet further is enabled to correlate to determined deviation with prescriptive guidance stored in guidance data store 250, required to correct the deviation through the use of neural network 270. Specifically, the prescriptive guidance can include, by way of example, the rotation of the ultrasound imaging probe 230 either in a clockwise or counter-clockwise direction, the movement of the ultrasound imaging probe 230 laterally away from the sternum, or medially towards the sternum, and the aiming of the imaging beam downwards or upwards by tiling the tail of the ultrasound imaging probe 230 upwards or downwards, respectively, to name only a few examples. As well, the prescriptive guidance can be NULL to the extent that the deviation is ideal, or the prescriptive guidance can be START OVER to the extent that the deviation is so great as to be expressed as indeterminate. Finally, the program code is yet further enabled to display the correlated remedial measures in the display of the host computing system 210.

In even further illustration of the operation of the prescriptive guidance module 300, FIG. 3 is a flow chart illustrating a process for prescriptively guiding an operator of an ultrasound imaging probe. Beginning in block 310, a view is specified for imaging a target organ and in block 320, a video clip image is acquired of the target organ according to the specified view. In block 330, a quality is determined for the acquired video clip image according to the specified view. In this regard, the quality of the video clip image may be determined, for instance, by submitting the video clip image to a neural network trained to determine a quality of a video clip for a specified view of a target organ, or by comparing the video clip image to a set of known video clips of known quality during a content-based image retrieval operation.

In block 340, prescriptive guidance is then computed that correlates to the determined quality including a determined deviation of the pose of the ultrasound imaging probe from ideal and a probability that a particular movement of the ultrasound imaging probe will improve the resultant video clip image towards an ideal form. In this regard, the prescriptive guidance includes one or more movements known to improve quality of an image of the specified view from the determined quality. In decision block 350, if the prescriptive guidance has associated therewith a threshold probability of improving the resultant video clip image towards an ideal form, the prescriptive guidance in block 360 is added to a first in first out queue of fixed size such that a least recently inserted entry in the queue is ejected from the queue as the prescriptive guidance is added to the queue.

In decision block 370, it is determined if a threshold number of entries in the queue exist for the same type of prescriptive guidance. If so, in block 380 an icon corresponding to the prescriptive guidance for which a threshold number of entries exist in the queue, is displayed in a user interface to the ultrasound diagnostic system. In this way. But, otherwise, in block 390, no guidance is displayed in the user interface. Thereafter, the process repeats in block 320. In this way, prescriptive guidance is presented in the user interface to the operator of the ultrasound diagnostic probe only when it is clear that the prescriptive guidance has a threshold probability of improving the resultant video clip image and such a determination of the prescriptive guidance has occurred a threshold number of times within a limited time span so as to provide confidence in the prescriptive guidance.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Computer readable program instructions described herein can be downloaded to respective computing/processing devices from a computer readable storage medium or to an external computer or external storage device via a network. The computer readable program instructions may execute entirely on the user's computer, partly on the user's computer, as a stand-alone software package, partly on the user's computer and partly on a remote computer or entirely on the remote computer or server. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Finally, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

We claim:

1. A method for prescriptively guiding an operator of an ultrasound imaging probe, the method comprising:
acquiring a sequence of image clips of a target organ of a subject utilizing an ultrasound imaging probe of an ultrasound diagnostic system;
determining a particular view of the target organ expressed by the acquired image clips;
assessing a quality of each of the acquired image clips in respect to the particular view; and,
on condition that the assessed quality of the acquired image clips fall below a pre-determined satisfactory quality for the particular view, computing in memory of the ultrasound diagnostic system for each of the acquired image clips a prescriptive movement of the ultrasound imaging probe pre-determined to produce quality improvement of a subsequently acquired image clip of the target organ for the particular view, and in response to a determination that the prescriptive movement has been computed a threshold number of times in connection with the sequence of image clips, displaying the prescriptive movement in a user interface of the ultrasound diagnostic system concurrently with a display in the user interface of subsequently acquired image clips of the target organ.

2. The method of claim 1, further comprising:
inserting each computed prescriptive movement for a corresponding one of the acquired image clips into a first in first out queue of fixed size; and,
displaying the prescriptive movement in the user interface in response to a determination that the prescriptive movement is present in a threshold number of entries of the first in first out queue.

3. The method of claim 2, wherein the inserting of a computed prescriptive movement occurs only when a probability associated with the computed prescriptive movement exceeds a threshold value.

4. The method of claim 1, wherein the selected one of the prescriptive movement is shown as a directive to rotate the ultrasound imaging probe without changing position of the ultrasound imaging probe.

5. The method of claim 1, wherein the selected one of the prescriptive movement is a changing of position of the ultrasound imaging probe in a specified direction.

6. The method of claim 1, wherein the selected one of the prescriptive movement is a changing of a pitch of the ultrasound imaging probe without changing position of the ultrasound imaging probe.

7. A data processing system configured for prescriptively guiding an operator of an ultrasound imaging probe, the system comprising:
a computer with memory and at least one processor;
a display coupled to the computer;
beamformer circuitry coupled to the computer and the display;
an ultrasound imaging probe comprising a transducer connected to the beamformer circuitry; and,
a prescriptive guidance module executing in the memory of the computer, the module comprising program code enabled upon execution by the processor of the computer to perform:
acquiring a sequence of image clips of a target organ of a subject utilizing an ultrasound imaging probe of an ultrasound diagnostic system;
determining a particular view of the target organ expressed by the acquired image clips;
assessing a quality of each of the acquired image clips in respect to the particular view; and,
on condition that the assessed quality of the acquired image clips fall below a pre-determined satisfactory quality for the particular view, computing in memory of the ultrasound diagnostic system for each of the acquired image clips a prescriptive movement of the ultrasound imaging probe pre-determined to produce quality improvement of a subsequently acquired image clip of the target organ for the particular view, and in response to a determination that the prescriptive movement has been computed a threshold number of times in connection with the sequence of image clips, displaying the prescriptive movement in a user interface of the ultrasound diagnostic system concurrently with a display in the user interface of subsequently acquired image clips of the target organ.

8. The system of claim 7, wherein the program code of the module is further enabled to perform:
inserting each computed prescriptive movement for a corresponding one of the acquired image clips into a first in first out queue of fixed size; and,
displaying the prescriptive movement in the user interface in response to a determination that the prescriptive movement is present in a threshold number of entries of the first in first out queue.

9. The system of claim 8, wherein the inserting of a computed prescriptive movement occurs only when a probability associated with the computed prescriptive movement exceeds a threshold value.

10. The system of claim 7, wherein the selected one of the prescriptive movement is shown as a directive to rotate the ultrasound imaging probe without changing position of the ultrasound imaging probe.

11. The system of claim 7, wherein the selected one of the prescriptive movement is a changing of position of the ultrasound imaging probe in a specified direction.

12. The system of claim 7, wherein the selected one of the prescriptive movement is a changing of a pitch of the ultrasound imaging probe without changing position of the ultrasound imaging probe.

13. A computer program product for prescriptively guiding an operator of an ultrasound imaging probe, the computer program product including a computer readable storage medium having program instructions embodied therewith, the program instructions executable by a device to cause the device to perform a method including:
acquiring a sequence of image clips of a target organ of a subject utilizing an ultrasound imaging probe of an ultrasound diagnostic system;
determining a particular view of the target organ expressed by the acquired image clips;
assessing a quality of each of the acquired image clips in respect to the particular view; and,
on condition that the assessed quality of the acquired image clips fall below a pre-determined satisfactory quality for the particular view, computing in memory of the ultrasound diagnostic system for each of the acquired image clips a prescriptive movement of the ultrasound imaging probe pre-determined to produce quality improvement of a subsequently acquired image clip of the target organ for the particular view, and in response to a determination that the prescriptive movement has been computed a threshold number of times in connection with the sequence of image clips, displaying the prescriptive movement in a user interface of the ultrasound diagnostic system concurrently with a display in the user interface of subsequently acquired image clips of the target organ.

14. The computer program product of claim 13, wherein the method further comprises:
inserting each computed prescriptive movement for a corresponding one of the acquired image clips into a first in first out queue of fixed size; and,
displaying the prescriptive movement in the user interface in response to a determination that the prescriptive movement is present in a threshold number of entries of the first in first out queue.

15. The computer program product of claim 14, wherein the inserting of a computed prescriptive movement occurs only when a probability associated with the computed prescriptive movement exceeds a threshold value.

16. The computer program product of claim 13, wherein the selected one of the prescriptive movement is shown as a directive to rotate the ultrasound imaging probe without changing position of the ultrasound imaging probe.

17. The computer program product of claim 13, wherein the selected one of the prescriptive movement is a changing of position of the ultrasound imaging probe in a specified direction.

18. The computer program product of claim 13, wherein the selected one of the prescriptive movement is a changing of a pitch of the ultrasound imaging probe without changing position of the ultrasound imaging probe.
